SEQUENCE LISTING (28 sequences)

SEQ ID NO: 1 - protein, 328 aa, Agrocybe aegerita
  mat_peptide (1)..(328)
EPGLPPGPLENSSAKLVNDEAHPWKPLRPGDIRGPCPGLNTLASHGYLPRNGVATPVQIINAVQEGLNFDNQAAVFATYAAHLVDGNLITDLLSIGRKTRLTGPDPPPPASVGGLNEHGTFEGDASMTRGDAFFGNNHDFNETLFEQLVDYSNRFGGGKYNLTVAGELRFKRIQDSIATNPNFSFVDFRFFTAYGETTFPANLFVDGRRDDGQLDMDAARSFFQFSRMPDDFFRAPSPRSGTGVEVVIQAHPMQPGRNVGKINSYTVDPTSSDFSTPCLMYEKFVNITVKSLYPNPTVHVRKALNTNLDFFFQGVAAGCTQVFPYGRD

SEQ ID NO: 2 - protein, 344 aa, Coprinopsis cinerea
  mat_peptide (1)..(344)
TSKLPIVFPPPPPEPIKDPWLKLVNDRAHPWRPLRRGDVRGPCPGLNTLASHGYLPRDGVATPAQIITAVQEGFNMEYGIATFVTYAAHLVDGNPLTNLISIGGKTRKTGPDPPPPAIVGGLNTHAVFEGDASMTRGDFHLGDNFNFNQTLWEQFKDYSNRYGGGRYNLTAAAELRWARIQQSMATNGQFDFTSPRYFTAYAESVFPINFFTDGRLFTSNTTAPGPDMDSALSFFRDHRYPKDFHRAPVPSGARGLDVVAAAYPIQPGYNADGKVNNYVLDPTSADFTKFCLLYENFVLKTVKGLYPNPKGFLRKALETNLEYFYQSFPGSGGCPQVFPWGKSD

SEQ ID NO: 3 - protein, 351 aa, Coprinopsis cinerea
MVSCKLPLPLLTLAIALANVNAFPAYQSLGGLSKRQLETIIPGLPVVNPGPPPGPLADSTLKLVNDAAHPYQAPRPHLDHRGPCPGLNTLANHGYLPRSGIATPAQIVQAVMEGFNMENTFAKFVTYAAFLVDGNPITNLMSIGGKTWRTGIIEPPPPAIVGGLNTHAVFEGDTSMTRGDFHFGDNHSFNQTLFDQFVEYSNIHGGGFYNLTAATELRYQRIQQSIATNPEMSFVSPRWFTAILLQDEKFPDDFHRAPGPFSFEGLGYLVTRRPMPPGRNVGGVDNYVPDPNSADFNSFCKMYEDFVNDIVVALYPNPTGLLRRNLIKNLEYFWTGMFDPACTEVKPYGTL

SEQ ID NO: 4 - protein, 243 aa, Aspergillus niger
LATGSTKLLPWSPPGHGDVRGPCPMLNTLANHGLLPHNGKDISQEVITEVLNNTLNLADGLSAFLFEEAMTTVEDPKATTFSLSDLNCPGILEHDGSLSRQDTYFGNNHEFNQTIFDQTKSYWTTPLIDMYQAAEAHEARLNTSKATNPTFNLSETGLTFSFGETAAYMIVFEDTNLGYANRSWVEYFFENERLPQELGWTKRPFITTGQVLVDMTTWVINSTIGVTPEEQAEMQDFGKKITG

SEQ ID NO: 5 - protein, 259 aa, Aspergillus niger
LATGSTKLLPWSPPGHGDVRGPCPMLNTLANHGLLPHNGKDISQEVITEVLNNTLNLADGLSAFLFEEAMTTVEDPKATTFSLSDLNCPGILEHDGSLRYLPLQKHRGNQADLLSRQDTYFGNNHEFNQTIFDQTKSYWTTPLIDMYQAAEAHEARLNTSKATNPTFNLSETGLTFSFGETAAYMIVFEDTNLGYANRSWVEYFFENERLPQELGWTKRPFITTGQVLVDMTTWVINSTIGVTPEEQAEMQDFGKKITG

SEQ ID NO: 6 - protein, 247 aa, Aspergillus niger
FPQQGAPHPLPWSPPGPNDVRAPCPMLNTLANHGYLPHNGKDITERHTINALYNALGIEEELAIYLHQEAVTTNPAPNATTFSLNDLSRHDILEHDASLSRQDAYFGDNHDFNQTIFDETRSYWTSPIIDVKQAAVSRQARVNTSMATNPNYTMSELGDSFSYGETAAYIIVLGDKEKGLVNRSRVEYLFENERLPLDLGWSRAKENITFDDLSTMLQRIINATGGEMDFRATIALPRLVYIYYEEA

SEQ ID NO: 7 - protein, 247 aa, Aspergillus niger
FPQQGAPHPLPWSPPGPNDVRAPCPMLNTLANHGYLPHNGKDITERHTINALYNALGIEEELAIYLHQEAVTTNPAPNATTFSLNDLSRHDILEHDASLSRQDAYFGDNHDFNQTIFDETRSYWTSPIIDVKQAAVSRQARVNTSMATNPNYTMSELGDSFSYGETAAYIIVLGDKEKGLVNRSRVEYLFENERLPLDLGWSRAKENITFDDLSTMLQRIINATGGESEFDRELAKRGGVHVGSWRG

SEQ ID NO: 8 - protein, 259 aa, Poronia punctata
KAACPYGYGEFQPEQTSDARGPCPVLNTLANHGYLPRDGRHIDENRTLTALHDALNLDIDFGKFLFTAGRLSNPKANSTWFDLDHLSRHGIFEHDGSLSRQDHHFGEWSRFNQTVWNWTLEYLPDDMLDVQTVANARAQRMTRSNLTNPDFALSYLGYLFSVGEAAAVLSILGDKKTQTCPKAFADYIFVNERLPYELGWKKQDASISFDDLVETFEDLERHTSFPFPPPLDNSTDIFDQLVEGGQSKKKRCSAHIGCF

SEQ ID NO: 9 - protein, 259 aa, Chaetomium virescens
ELDFSKWKTRQPGEFRAPCPAMNSLANHGFIPRDGRNITVAMLVPVLQEVFHLSPELAQTISTLGLFTAQDPSKGVFTLDDLNRHNLFEHDASLSREDYYFHKDASTFRPEVFKKFMSHFKGKEYVTLEDAASARYAMVQESRKKNPTFTYTVQQRITSYGETIKYFRTIVEPATGKCPVAWIKILFEQERLPYNEGWRPPKAELSGFSMASDVLELALVTPEKLIDKPCEGKQCPQARGIHGYFGMLLPITAQELAVK

SEQ ID NO: 10 - protein, 247 aa, Chaetomium virescens
ELDFSKWKTRQPGEFRAPCPAMNSLANHGFIPRDGRNITVAMLVPVLQEVFHLSPELAQTISTLGLFTAQDPSKGVFTLDDLNRHNLFEHDASLSREDYYFHKDASTFRPEVFKKFMSHFKGKEYVTLEDAASARYAMVQESRKKNPTFTYTVQQRITSYGETIKYFRTIVEPATGKCPVAWIKILFEQERLPYNEGWRPPKAELSGFSMASDVLELALVTPEKLIDKPCEGKQCPQARGIHGYFGM

SEQ ID NO: 11 - protein, 261 aa, Humicola insolens
GFHDWEPPGPNDVRAPCPMLNTLANHGFLPHHGRDLTRKQVVDGLYNGLNINKTAASTLFDFALMTSPKPNATTFSLDDLGRHNILEHDASLSRTDAYFGDVLAFNKTIFEETKRHWGKSPILDVTAAARARLGRIQTSKATNPEYFMSELGNIFTYGESVAYIMLIGDAKTGRANRRWVEYWFENERLPTHLGWRRPSKELTSDVLDTYISLIQNITLTLPGGTDPVKRRAASHFVFPFGQGLGGPAGVALMLISVVAYG

SEQ ID NO: 12 - protein, 238 aa, Humicola insolens
GFHDWEPPGPNDVRAPCPMLNTLANHGFLPHHGRDLTRKQVVDGLYNGLNINKTAASTLFDFALMTSPKPNATTFSLDDLGRHNILEHDASLSRTDAYFGDVLAFNKTIFEETKRHWGKSPILDVTAAARARLGRIQTSKATNPEYFMSELGNIFTYGESVAYIMLIGDAKTGRANRRWVEYWFENERLPTHLGWRRPSKELTSDVLDTYISLIQNITLTLPGGTDPVKRRAASHFGW

SEQ ID NO: 13 - protein, 242 aa, Chaetomium globosum
GFDTWAPPGPYDVRGPCPMLNTLTNHGFFPHDGQDIDRETTENALFDALHVNKTLASFLFDFALTTNPIANSTTFSLNDLGNHNVLEHDASLSRADAYHGSVLAFNHTIFEETKSYWTDETVTLKMAADARYYRIKSSQATNPTYQMSELGDAFTYGESAAYVVLFGDKESQTVPRSWVEWLFEKEQLPQHLGWKRPATSFELNDLDKFMALIQNYTQEIEEPSCESRKQRRKPRGPSHFGF

SEQ ID NO: 14 - protein, 301 aa, Chaetomium globosum
GFDTWAPPGPYDVRAPCPMLNTLANHGFLPHDGHEITREQTENALFDALHIDKMLGSSLFDFAMTTNPVANSTTFSLNDLGNHNVLEHDASLSRSDAYFGNTLTFNQTVFDETKSYWTDETVTIEMASNARLARIKTSNATNPTYSMSELGNGFTKGESAAYVVIFGDKISGTVPRAWVEWLFEHEQLPQHLGWKRPTELFRDGDLDKYMDAMQNVIVEIALKTQPSTPSIKPTQTPSSPTRLLLKRLGRQLMLIVPRPIRLRVLRNTPPLRLITKNKPREMAPNLLILAVHKRATSMQKR

SEQ ID NO: 15 - protein, 344 aa, Chaetomium globosum
GFDTWAPPGPYDVRAPCPMLNTLANHGFLPHDGHEITREQTENALFDALHIDKMLGSSLFDFAMTTNPVANSTTFSLNDLGNHNVLEHDASLSRSDAYFGNTLTFNQTVFDETKSYWTDETVTIEMASNARLARIKTSNATNPTYSMSELGNGFTKGESAAYVVIFGDKISGTVPRAWVEWLFEHEQLPQHLGWKRPTELFRDGDLDKYMDAMQNVIVGETPGCPAGKQQQRKGRRTPSHFGWDPTPVNKLASLRIAGYCHEIALKTQPSTPSIKPTQTPSSPTRLLLKRLGRQLMLIVPRPIRLRVLRNTPPLRLITKNKPREMAPNLLILAVHKRATSMQKR

SEQ ID NO: 16 - protein, 243 aa, Chaetomium globosum
GFDTWAPPGPYDVRAPCPMLNTLANHGFLPHDGHEITREQTENALFDALHIDKMLGSSLFDFAMTTNPVANSTTFSLNDLGNHNVLEHDASLSRSDAYFGNTLTFNQTVFDETKSYWTDETVTIEMASNARLARIKTSNATNPTYSMSELGNGFTKGESAAYVVIFGDKISGTVPRAWVEWLFEHEQLPQHLGWKRPTELFRDGDLDKYMDAMQNVIVGETPGCPAGKQQQRKGRRTPSHFGW

SEQ ID NO: 17 - protein, 276 aa, Sclerotinia sclerotiorum
MKLNFLSTTLALGLVSARAHYQQQIIANDTEGEWIAPSATDYRGPCPMLNTLANHGFLPRDGRNLTEHNVVKGLNHGLNFNKSLGSIMFQHAVPASPAYPNTTFFTLDDLNRHNVLEHDASISRSDAYFGNNHIFNQTIFDTTKMYWPSETLTAQHLIDGKIFRQIVSRTTNPNYTFTSTTQAFSLGEMAAPIVAFGDKNALTANRTLVESWIENERLPTELGWSKPEEEVSLGDILYVTGALANLTSLLSDVVITPRGESAGAHAKRMGHWGVSM

SEQ ID NO: 18 - protein, 259 aa, Aspergillus carbonarius
MKSTILLITTSLSQALAQVSSHPFPWSAPGPNDVRGPCPMLNTLANHGFLPHDGKDITEDRIVMVLNNSLNLDEELSQFLFKEALTTNPDPNATTFSLNDLSRHNILEHDASLSRQDYYFGDNHDFNQTVFNETRSYWTAPLIDFNAAAQARLARVNTSMATNPTYTESETGLAFSYGESAAYMIVFAEGSETANRSWVEYFFEHERLPQQLGWTKPQESISSSVLIDTVTGIANASNASSLVVAELLDFVALHMGRLP

SEQ ID NO: 19 - protein, 838 aa, Glycine max
FSAGHKIKGTVVLMPKNELEVNPDGSAVDNLNAFLGRSVSLQLISATKADAHGKGKVGKDTFLEGINTSLPTLGAGESAFNIHFEWDGSMGIPGAFYIKNYMQVEFFLKSLTLEAISNQGTIRFVCNSWVYNTKLYKSVRIFFANHTYVPSETPAPLVSYREEELKSLRGNGTGERKEYDRIYDYDVYNDLGNPDKSEKLARPVLGGSSTFPYPRRGRTGRGPTVTDPNTEKQGEVFYVPRDENLGHLKSKDALEIGTKSLSQIVQPAFESAFDLKSTPIEFHSFQDVHDLYEGGIKLPRDVISTIIPLPVIKELYRTDGQHILKFPQPHVVQVSQSAWMTDEEFAREMIAGVNPCVIRGLEEFPPKSNLDPAIYGDQSSKITADSLDLDGYTMDEALGSRRLFMLDYHDIFMPYVRQINQLNSAKTYATRTILFLREDGTLKPVAIELSLPHSAGDLSAAVSQVVLPAKEGVESTIWLLAKAYVIVNDSCYHQLMSHWLNTHAAMEPFVIATHRHLSVLHPIYKLLTPHYRNNMNINALARQSLINANGIIETTFLPSKYSVEMSSAVYKNWVFTDQALPADLIKRGVAIKDPSTPHGVRLLIEDYPYAADGLEIWAAIKTWVQEYVPLYYARDDDVKNDSELQHWWKEAVEKGHGDLKDKPWWPKLQTLEDLVEVCLIIIWIASALHAAVNFGQYPYGGLIMNRPTASRRLLPEKGTPEYEEMINNHEKAYLRTITSKLPTLISLSVIEILSTHASDEVYLGQRDNPHWTSDSKALQAFQKFGNKLKEIEEKLVRRNNDPSLQGNRLGPVQLPYTLLYPSSEEGLTFRGIPNSISI

SEQ ID NO: 20 - protein, 8 aa, Artificial
  Motif sequence
EHDXSXSR

SEQ ID NO: 21 - protein, 247 aa, Penicillium chrysogenum
SPGAGWKHALQWKPAGESDYRGPCPMMNTLANHGFLPHDGRNITRPNLVDALGQALNFNGTLASLMFDMGVVANPEPNATVFTLDDLNRHNVLEHDASLSRSDAFFGSNHVFNETIFEETKAYWTGPILDAEMLANSKVARQINSKAHNPTYTFTANTEQFSLGEVAAPIIAFGDIQAGTVNRSLVEYFFENERLPTDLGWKRPAKVTSLQDILSVTQMIKKASRLITPSESSPARHQGSSQVNLHG

SEQ ID NO: 22 - protein, 244 aa, Daldinia caldariorum
APWKAPGPDDVRGPCPMLNTLANHGFLPHDGKNIDVNTTVNALSSALNLDDELSRDLHTFAVTTNPQPNATWFSLNHLSRHNVLEHDASLSRQDAYFGPPDVFNAAVFNETKAYWTGDIINFQMAANALTARLMTSNLTNPEFSMSQLGRGFGLGETVAYVTILGSKETRTVPKAFVEYLFENERLPYELGFKKMKSALTEDELTTMMGEIYSLQHLPESFTKPFAKRSEAPFEKRAEKRCPFH

SEQ ID NO: 23 - protein, 244 aa, Myceliophthora fergusii
GFDTWSPPGPYDVRAPCPMLNTLANHGFLPHDGKDITREQTENALFEALHINKTLGSFLFDFALTTNPRNTSTFSLNDLGNHNILEHDASLSRADAYFGNVLQFNQTVFDETKTYWDGDVIDLRMAARARLGRIKTSQATNPTYSMSELGDAFTYGESAAYVVVLGDKESRTAKRSWVEWFFEHEQLPQHLGWKRPASSLEEEDLFTIMDEIRQYTSELEGSTSSSDAQTSRRQLPRRRTHFGF

SEQ ID NO: 24 - protein, 244 aa, Myceliophthora hinnulea
GFDTWSPPGPYDVRAPCPMLNTLANHGFLPHDGKDITREQTENALFDALNINKTLASFLFDFALTTNPKNTSTFSLNDLGNHNILEHDASLSRADAYFGNVLQFNQTVFDETKTYWEGDTIDLRMAAKARLGRIKTSQATNPTYSMSELGDAFTYGESAAYVVVLGDKESRTVNRSWVEWFFEHEQLPQHLGWKRPAVSFEEEDLNRFMEEIEKYTKGLEGSNSTSGSQKHRRRLPRRRTHFGF

SEQ ID NO: 25 - protein, 249 aa, Thielavia terrestris
SPDWSSPEFWSWHPPAPGDDRRGPCPMLNTLANHGFLPHNGRNITKEITVNALNSALNVNKTLGELLFNFAVTTNPQPNATFFDLDHLSRHNILEHDASLSRADYYFGHDDHTFNQTVFDQTKSYWKTPIIDVQQAANARLARVLTSNATNPTFVLSQIGEAFSFGETAAYILALGDRVSGTVPRQWVEYLFENERLPLELGWRRAKEVISNSDLDQLTNRVINATGALANITRKIKVRDFHAGRFPGE

SEQ ID NO: 26 - protein, 241 aa, Thielavia hyrcaniae
GFDTWSPPGPYDVRAPCPMLNTLANHGFLPHDGKDLTRDVVENALSDALNINKTLGSFLFDFALTTNPKPNSTTFSLNDLGNHNILEHDASLSRSDAYFGNVLVFNQSVFDETKSYFKGKTVTLKQAAQARLARIKTSKATNPTYSMSQLGDSFTYGESAAYVVVLGGDKKSATVPRSWVEWFFEHEQLPQHLGWKRPAEQFTQDDLNKFMDEIINITKAIDHRSVDVPEKTKRRISHWGA

SEQ ID NO: 27 - protein, 237 aa, Pestalotiopsis virgatula
DFDTWSPPGPDDVRAPCPMLNSLANHGFFPHDGKDITEDVTIAALADALNVDKSLSQFLHDKAVSTNPTPGATTFSLSDLSNHNILEHDASLSRADYYWGDDHTFNETVFNETRSYWTDETVTVKMAADARLARVHSSIATNPSYSMSDLGNEFSLGETAAYIIALGDRDDATVQKSFVEYLFENERLPLELGWARPEELIDLGDVQDMLFRVIDATDSDAATMAKLRKRGGYHAGL

SEQ ID NO: 28 - protein, 246 aa, Pestalotiopsis virgatula
DGCSNYSVPEWHPPTEGDVRGPCPMLNTLANHGYLPHSGKDINVNKTIDALGQALNIDAELATFLHSFAVTTNPTPNATIFSLDNLSRHNILEHDGSLSRADYYWTGDATSFNQTVFDETRSYWTTPIIDMEQAAAARVARMQTSQATNPNFTLSDLGSAFSIGESAAYIFILGDRVSGTVERSLVEYLFENERLPTALGWKRAAESISEDDLADAMDRIVNATNTNTTAGTSKRGINGRARLPRL

(19) United States
(12) Patent Application Publication
(10) Pub. No.: US 2015/0184208 A1
(43) Pub. Date: Jul. 2, 2015

(54) ENZYMATIC REDUCTION OF HYDROPEROXIDES

(71) Applicant: Novozymes A/S, Bagsvaerd (DK)

(72) Inventors: Lars Oestergaard, Charlottelund (DK); Lisbeth Kalum, Vaerloese (DK); Henrik Lund, Vaerloese (DK)

(21) Appl. No.: 14/408,696

(22) PCT Filed: Jun. 12, 2013
(86) PCT No.: PCT/EP2013/062124

§ 371 Date: Dec. 17, 2014

Related U.S. Application Data
(60) Provisional application No. 61/665,345, filed on Jun. 28, 2012.

(30) Foreign Application Priority Data
Jun. 19, 2012 (EP) 12172658.2

(51) Int. Cl. C12P 7/64; C12N 9/02; C11D 3/386; C12N 9/08

(52) U.S. Cl. CPC C12P 7/6409; C12N 9/0065; C12N 9/0069; C11D 3/38654; C12Y 111/02001; C12Y 113/11012

(57) ABSTRACT

The invention relates to enzymatic methods for reducing the amount of fatty acid hydroperoxides in a fatty acid containing fat/oil product.

### REFERENCE TO A SEQUENCE LISTING

This application contains a Sequence Listing in computer readable form. The computer readable form is incorporated herein by reference.

### BACKGROUND OF THE INVENTION

1. Field of the Invention

The present invention relates to use of peroxygenases for reducing the amount of fatty acid hydroperoxides in an oil/fat product.

2. Background

The peroxide number is a measurement of the concentration of (—O—O—) groups in edible oils. It is a measurement of the decomposition of the fat/oil product, and in many countries official standards specify a maximum peroxide number beyond which the oil is unfit for human consumption. The peroxide number is therefore measured by oil manufacturers during production and after storage to check its preservation.

International standards use a redox titration in non-aqueous media, and results are generally expressed in pg of peroxide (or active oxygen) per gram of product but mmoles/kg or meq of O₂/kg are also used.

Therefore, a treatment of fat and oil products with a peroxygenase, as claimed below, can serve as a quality upgrade of the product, by reducing the peroxide number.

In the specific case of unsaturated fat and oil products the combined process of treatment with lipoxygenase and peroxygenase may be used as a mean to produce a natural oil polyol that can be used as raw material for a variety of chemical processes.

Belikova et al. “Heterolytic reduction of fatty acid hydroperoxides by cytochrome c/cardiolipin complexes: antioxidant function in mitochondria”, J. Am. Chem. Soc., (2009) 131 (32), pp 11288-11289 discloses related reactions in mitochondria.

### SUMMARY OF THE INVENTION

The inventors of the present invention have surprisingly found that peroxygenases can use fatty acid hydroperoxides as a source of oxidizing power as an alternative to hydrogen peroxide. Accordingly, peroxygenases can serve as a tool to reduce the content of fatty acid hydroperoxides from fat/oil products, which will allow for quality upgrading of the fat/oil product.

The oxygen from a fatty acid hydroperoxide can be transferred to the same or another fatty acid in the fat/oil product as e.g., a hydroxyl group (see for example WO 2011/120938).

It is to be understood that in the context of this invention, a fatty acid and an acyl group of a lipid are equivalents.

In a particular embodiment, the fatty acid hydroperoxide can be the product from the reaction between an unsaturated fatty acid and molecular oxygen catalyzed by a lipoxygenase. Thereby the treatment of an unsaturated fat/oil product with a combination of lipoxygenase and peroxygenase will enable the direct conversion of a fat/oil product into a polyol without the addition of hydrogen peroxide.

Accordingly, in a first aspect, the present invention provides a method for reducing the amount of fatty acid hydroperoxides in a fatty acid containing product, comprising contacting the fatty acid hydroperoxides with a peroxygenase.

In another aspect, the invention provides a composition for making a fatty acid polyol, comprising a peroxygenase and a lipoxygenase; and a method for making a fatty acid polyol, comprising contacting a fatty acid with a peroxygenase, a lipoxygenase and oxygen.

In yet another aspect, the invention provides a method and composition for bleaching a carotenoid containing dye, stain, or composition, comprising contacting the carotenoid with a peroxygenase, a lipoxygenase and oxygen.

In embodiments, the peroxygenase comprises an amino acid sequence which has at least 60% identity to SEQ ID NO: 1, 2, 3, 4, 5, 6, 7, 8, 9, 10, 11, 12, 13, 14, 15, 16, 17, 18, 21, 22, 23, 24, 25, 26, 27, or 28. In other embodiments, the amino acid sequence comprises the motif: E-H-D-[G,A]-S-[L,I]-S-R.

Other aspects and embodiments of the invention are apparent from the description and examples.

### Definitions

Peroxygenase: The term “peroxygenase” means an enzyme exhibiting “unspecific peroxygenase” activity according to EC 1.11.2.1, that catalyzes insertion of an oxygen atom from H₂O₂ into a variety of substrates, such as nitrobenzodioxole. For purposes of the present invention, peroxygenase activity is determined according to the procedure described in M. Poraj-Kobielska, M. Kinne, R. Ullrich, K. Scheibner, M. Hofrichter, “A spectrophotometric assay for the detection of fungal peroxygenases”, Analytical Biochemistry (2012), vol. 421, issue 1, pp. 327-329.

The peroxygenase of the present invention has at least 20%, preferably at least 40%, more preferably at least 50%, more preferably at least 60%, more preferably at least 70%, more preferably at least 80%, even more preferably at least 90%, most preferably at least 95%, and even most preferably at least 100% of the peroxygenase activity of the mature polypeptide of SEQ ID NO: 1, 2, 3, 4, 5, 6, 7, 8, 9, 10, 11, 12, 13, 14, 15, 16, 17, 18, 21, 22, 23, 24, 25, 26, 27, or 28.

Mature polypeptide: The term “mature polypeptide” is defined herein as a polypeptide having peroxygenase activity that is in its final form following translation and any post-translational modifications, such as N-terminal processing, C-terminal truncation, glycosylation, phosphorylation, etc. In a preferred aspect, the mature polypeptide has the amino acid sequence shown in positions 1 to 328 of SEQ ID NO:1 based on the N-terminal peptide sequencing data (Ullrich et al., 2004, Appl. Env. Microbiol. 70(8): 4575-4581), elucidating the start of the mature protein of AaP peroxygenase enzyme.

Identity: The relatedness between two amino acid sequences or between two nucleotide sequences is described by the parameter “identity”.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; http://emboss.orq), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra; http://emboss.orq), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment).

Modification: The term “modification” means herein any chemical modification of the polypeptide consisting of the mature polypeptide of SEQ ID NO: 1, 2, 3, 4, 5, 6, 7, 8, 9, 10, 11, 12, 13, 14, 15, 16, 17, 18, 21, 22, 23, 24, 25, 26, 27, or 28; or a homologous sequence thereof; as well as genetic manipulation of the DNA encoding such a polypeptide. The modification can be a substitution, a deletion and/or an insertion of one or more (several) amino acids as well as replacements of one or more (several) amino acid side chains.

### DETAILED DESCRIPTION OF THE INVENTION

### Peroxygenase

The peroxygenase of the present invention is preferably recombinantly produced, and comprises or consists of an amino acid sequence having at least 70% identity, preferably at least 75%, 80%, 85%, 90%, 95%, 97%, 98%, or 99% identity to the amino acid sequence of SEQ ID NO: 1, 2, 3, 4, 5, 6, 7, 8, 9, 10, 11, 12, 13, 14, 15, 16, 17, 18, 21, 22, 23, 24, 25, 26, 27, or 28; preferably SEQ ID NO: 1, 2, 8, 9, 11, 13, 17, 22, or 23.

In a preferred embodiment, the peroxygenase comprises an amino acid sequence represented by the motif: E-H-D-[G,A]-S-[L,I]-S-R (SEQ ID NO:20).

In yet another embodiment, the peroxygenase of the first aspect comprises or consists of the amino acid sequence of SEQ ID NO: 1, 2, 3, 4, 5, 6, 7, 8, 9, 10, 11, 12, 13, 14, 15, 16, 17, 18, 21, 22, 23, 24, 25, 26, 27, or 28; preferably SEQ ID NO: 1, 2, 8, 9, 11, 13, 17, 22, or 23; or a fragment thereof having peroxygenase activity; preferably the polypeptide comprises or consists of the mature polypeptide of SEQ ID NO: 1, 2, 3, 4, 5, 6, 7, 8, 9, 10, 11, 12, 13, 14, 15, 16, 17, 18, 21, 22, 23, 24, 25, 26, 27, or 28; preferably SEQ ID NO: 1, 2, 8, 9, 11, 13, 17, 22, or 23. Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., peroxygenase activity) to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with polypeptides that are related to a polypeptide according to the invention.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

The total number of amino acid substitutions, deletions and/or insertions of the mature polypeptide of SEQ ID NO: 1, 2, 3, 4, 5, 6, 7, 8, 9, 10, 11, 12, 13, 14, 15, 16, 17, 18, 21, 22, 23, 24, 25, 26, 27, or 28; preferably SEQ ID NO: 1, 2, 8, 9, 11, 13, 17, 22, or 23; is at most 10, preferably at most 9, more preferably at most 8, more preferably at most 7, more preferably at most 6, more preferably at most 5, more preferably at most 4, even more preferably at most 3, most preferably at most 2, and even most preferably at most 1.

The concentration of peroxygenase is typically 0.05 mg/ml to 50 mg/ml, preferably 0.05 mg/ml to 10 mg/ml, more preferably 0.1 mg/ml to 10 mg/ml, and most preferably 0.1 mg/ml to 5 mg/ml.

### Lipoxygenase

The lipoxygenase (may be referred to as LOX) of the invention is a lipoxygenase, classified as EC 1.13.11.12, which is an enzyme that catalyzes the oxygenation of polyunsaturated fatty acids, especially cis,cis-1,4-dienes, e.g., linoleic acid and produces a hydroperoxide. But also other substrates may be oxidized, e.g., monounsaturated fatty acids.

Microbial lipoxygenases can be derived from, e.g., Saccharomyces cerevisiae, Thermoactinomyces vulgaris, Fusarium oxysporum, Fusarium proliferatum, Thermomyces lanuginosus, Pyricularia oryzae, and strains of Geotrichum. The preparation of a lipoxygenase derived from Gaeumannomyces graminis is described in Examples 3-4 of WO 02/20730. The expression in Aspergillus oryzae of a lipoxygenase derived from Magnaporthe salvinii is described in Example 2 of WO 02/086114, and this enzyme can be purified using standard methods, e.g. as described in Example 4 of WO 02/20730.

Lipoxygenase may also be extracted from plant seeds, such as soybean, pea, chickpea, and kidney bean. Alternatively, lipoxygenase may be obtained from mammalian cells, e.g. rabbit reticulocytes.

The lipoxygenase of the present invention is preferably recombinantly produced, and comprises or consists of an amino acid sequence having at least 70% identity, preferably at least 75%, 80%, 85%, 90%, 95%, 97%, 98%, or 99% identity to the amino acid sequence of SEQ ID NO: 19.

In an embodiment, the lipoxygenase comprises or consists of the amino acid sequence of SEQ ID NO: 19; or a fragment thereof having lipoxygenase activity; preferably the lipoxygenase comprises or consists of the mature polypeptide of SEQ ID NO: 19.

Preferably, amino acid changes are of a minor nature, that is conservative amino acid substitutions or insertions that do not significantly affect the folding and/or activity of the protein;

small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., peroxygenase activity) to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with polypeptides that are related to a polypeptide according to the invention.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

The total number of amino acid substitutions, deletions and/or insertions of the lipoxygenase of SEQ ID NO: 19 is at most 10, preferably at most 9, more preferably at most 8, more preferably at most 7, more preferably at most 6, more preferably at most 5, more preferably at most 4, even more preferably at most 3, most preferably at most 2, and even most preferably at most 1.

The concentration of lipoxygenase is typically 0.05 mg/ml to 50 mg/ml, preferably 0.05 mg/ml to 10 mg/ml, more preferably 0.1 mg/ml to 10 mg/ml, and most preferably 0.1 mg/ml to 5 mg/ml.

### Lipoxygenase Activity

Lipoxygenase activity may be determined spectrophotometrically at 25° C. by monitoring the formation of hydroperoxides. For the standard analysis, 10 micro liters enzyme is added to a 1 ml quartz cuvette containing 980 micro liter 25 mM sodium phosphate buffer (pH 7.0) and 10 micro liter of substrate solution (10 mM linoleic acid dispersed with 0.2% (v/v) Tween20). The enzyme is typically diluted sufficiently to ensure a turn-over of maximally 10% of the added substrate within the first minute. The absorbance at 234 nm is followed and the rate is estimated from the linear part of the curve. The cis-trans-conjugated hydro(pero)xy fatty acids are assumed to have a molecular extinction coefficient of 23,000 M⁻¹cm⁻¹.

### Oxygen

The oxygen required by the lipoxygenase may be oxygen from the atmosphere or an oxygen precursor for in situ production of oxygen. In many industrial applications, oxygen from the atmosphere will usually be present in sufficient quantity. If more O₂ is needed, additional oxygen may be added, e.g. as pressurized atmospheric air or as pure pressurized O₂.

### Fatty Acid

A fatty acid is a carboxylic acid with an aliphatic tail (chain), which is either saturated or unsaturated. Most naturally occurring fatty acids have a chain of an even number of carbon atoms, from 4 to 28. Fatty acids are usually derived from triglycerides or phospholipids. When they are not attached to other molecules, they are known as “free” fatty acids. Examples of fatty acids include, but are not limited to, butanoic acid (butyric acid), pentanoic acid (valeric acid), hexanoic acid (caproic acid), heptanoic acid (enanthic acid), octanoic acid (caprylic acid), nonanoic acid (pelargonic acid), decanoic acid (capric acid), dodecanoic acid (lauric acid), tetradecanoic acid (myristic acid), hexadecanoic acid (palmitic acid), octadecanoic acid (stearic acid), eicosanoic acid (arachidic acid), linoleic acid, linolenic acid, arachidonic acid, eicosapentaenoic acid, and docosahexaenoic acid.

It is to be understood that in the context of this invention, a fatty acid and an acyl group of a lipid are equivalents. When the fatty acid is an acyl group of a lipid, the lipid can be a monoglyceride, diglyceride, triglyceride, phospholipid or sphingolipid. The acyl group may be saturated or unsaturated, and optionally functional groups (substituents) may be attached. Examples of acyl groups include, but are not limited to, the acyl forms of butanoic acid (butyric acid), pentanoic acid (valeric acid), hexanoic acid (caproic acid), heptanoic acid (enanthic acid), octanoic acid (caprylic acid), nonanoic acid (pelargonic acid), decanoic acid (capric acid), dodecanoic acid (lauric acid), tetradecanoic acid (myristic acid), hexadecanoic acid (palmitic acid), octadecanoic acid (stearic acid), eicosanoic acid (arachidic acid), linoleic acid, linolenic acid, arachidonic acid, eicosapentaenoic acid, and docosahexaenoic acid.

### Carotenoids

Carotenoids are organic pigments. All carotenoids are tetraterpenoids, meaning that they are produced from 8 isoprene molecules and contain 40 carbon atoms. Carotenoids in general absorb blue light. They are split into two classes, xanthophylls (which contain oxygen) and carotenes (which are purely hydrocarbons, and contain no oxygen). The most common carotenoids include the carotenes, lycopene (for example from tomatoes) and β-carotene (for example from carrots).

Preferred carotenoids according to the invention are carotenes. More preferred carotenoids are lycopene and beta-carotene.

Carotenoid and carotene containing compositions are often strongly colored. Examples of carotene containing compositions are foods containing tomatoes (like ketchup) or carrots. Stains of such foods on textiles are difficult to clean.

The methods and compositions of the invention can be used to bleach (reduce) the color of carotene containing dyes, stains (like food stains on textiles and other surfaces), and compositions.

### Surfactants

The method of the invention may include application of a surfactant (for example, as part of a detergent formulation or as a wetting agent). Surfactants suitable for being applied may be non-ionic (including semi-polar), anionic, cationic and/or zwitterionic; preferably the surfactant is anionic (such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap) or non-ionic (such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”)), or a mixture thereof.

When included in the method of the invention, the concentration of the surfactant will usually be from about 0.01% to about 10%, preferably about 0.05% to about 5%, and more preferably about 0.1% to about 1% by weight.

### Detergent Composition

In one embodiment, the invention is directed to detergent compositions comprising a peroxygenase and a lipoxygenase, as described above, in combination with one or more additional cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

In one embodiment of the present invention, the peroxygenase and lipoxygenase enzymes may each be added to a detergent composition in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.

The enzyme(s) of the detergent composition of the invention may be stabilized using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708. A polypeptide of the present invention may also be incorporated in the detergent formulations disclosed in WO97/07202, which is hereby incorporated by reference.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized.

When included therein the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent will usually contain from about 0.1% to about 10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the detergent will usually contain from about 0.1% to about 20% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein the detergent will usually contain from about 0.1% to about 10% by weight of a zwitterionic surfactant. Non-limiting examples of zwitterionic surfactants include betaine, alkyldimethylbetaine, sulfobetaine, and combinations thereof.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also contain 0-50% by weight, such as about 5% to about 30%, of a detergent co-builder, or a mixture thereof. The detergent composition may include include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)-ethylidenediamine-N,N′,N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977.053.

### Bleaching Systems

The detergent may contain 0-50% of a bleaching system. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof.

Suitable preformed peracids include, but are not limited to, peroxycarboxylic acids and salts, percarbonic acids and salts, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO98/17767. A particular family of bleach activators of interest was disclosed in EP624154 and particulary preferred in that family is acetyl triethyl citrate (ATC). ATC or a short chain triglyceride like triacetin has the advantage that it is environmental friendly as it eventually degrades into citric acid and alcohol. Furthermore acetyl triethyl citrate and triacetin has a good hydrolytical stability in the product upon storage and it is an efficient bleach activator. Finally ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

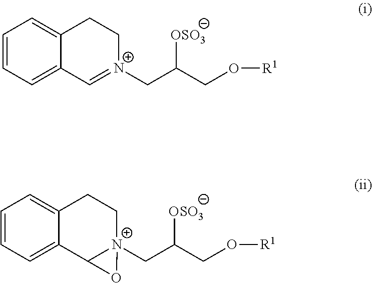

and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g. in WO2007/087258, WO2007/087244, WO2007/087259 and WO2007/087242. Suitable photobleaches may for example be sulfonated zinc phthalocyanine.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light.

Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### (Additional) Enzymes

The detergent composition may include one or more additional enzymes such as a protease, lipase, cutinase, amylase, carbohydrase, cellulase, pectinase, mannanase, arabinase, galactanase, and/or xylanase.

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include Celluzyme™, and Carezyme™ (Novozymes NS), Clazinase™, and Puradax HA™ (Genencor International Inc.), and KAC-500(B)™ (Kao Corporation).

Proteases: Suitable proteases include those of animal, vegetable or microbial origin. Microbial origin is preferred. Chemically modified or protein engineered mutants are included. The protease may be a serine protease or a metalloprotease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g., of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235, and 274.

Preferred commercially available protease enzymes include Alcalase™, Savinase™, Primase™, Duralase™, Esperase™, and Kannase™ (Novozymes NS), Maxatase™, Maxacal™, Maxapem™, Properase™, Purafect™, Purafect OxP™, FN2™, and FN3™ (Genencor International Inc.).

Lipases and Cutinases: Suitable lipases and cutinases include those of bacterial or fungal origin. Chemically modified or protein engineered mutants are included. Examples include lipase from Thermomyces, e.g., from T. lanuginosus (previously named Humicola lanuginosa) as described in EP 258 068 and EP 305 216, cutinase from Humicola, e.g. H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., 1993, Biochemica et Biophysica Acta, 1131: 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079, WO 97/07202, WO 00/060063, WO2007/087508 and WO 2009/109500.

Preferred commercially available lipase enzymes include Lipolase™, Lipolase Ultra™, and Lipex™; Lecitase™, Lipolex™; Lipoclean™, Lipoprime™ (Novozymes NS). Other commercially available lipases include Lumafast (Genencor Int Inc); Lipomax (Gist-Brocades/Genencor Int Inc) and Bacillus sp lipase from Solvay.

Amylases: Suitable amylases (α and/or β) include those of bacterial or fungal origin. Chemically modified or protein engineered mutants are included. Amylases include, for example, α-amylases obtained from Bacillus, e.g., a special strain of Bacillus licheniformis, described in more detail in GB 1,296,839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are Duramyl™, Termamyl™, Fungamyl™ and BAN™ (Novozymes NS), Rapidase™ and Purastar™ (from Genencor International Inc.).

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants—The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents—The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent—The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino)stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino)stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

Soil release polymers—The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents—The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multi compartments. It can be of any form, shape and material which is suitable for holding the composition, e.g. without allowing release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may also be non-aqueous.

### Methods, Compositions and Uses

In a first aspect, the present invention provides a method for reducing the amount of fatty acid hydroperoxides in a fatty acid containing product, comprising contacting the fatty acid hydroperoxide with a peroxygenase.

In a second aspect, the present invention provides a method for hydroxylating an unsaturated fatty acid, comprising contacting the unsaturated fatty acid with a lipoxygenase, oxygen, and a peroxygenase.

In a third aspect, the present invention provides a method for bleaching the color of a carotenoid containing dye, stain (like food stains on textiles and other surfaces), or composition, comprising contacting the carotenoid with a lipoxygenase, oxygen, and a peroxygenase. Preferably, the carotenoid is a carotene; more preferably the carotenoid is a lycopene (for example, derived from tomatoes or ketchup) or beta-carotene (for example, derived from carrots). Thus, in an embodiment, the carotenoid containing dye, stain, or composition is a tomato containing dye, stain, or composition; or a carrot containing dye, stain, or composition.

In embodiments, the peroxygenase comprises an amino acid sequence which has at least 70% identity to SEQ ID NO: 1, 2, 3, 4, 5, 6, 7, 8, 9, 10, 11, 12, 13, 14, 15, 16, 17, 18, 21, 22, 23, 24, 25, 26, 27, or 28; preferably SEQ ID NO: 1, 2, 8, 9, 11, 13, 17, 22, or 23.

In other embodiments, the amino acid sequence of the peroxygenase comprises the motif: E-H-D-[G,A]-S-[L,I]-S-R (SEQ ID NO: 20).

In other embodiments, the peroxygenase comprises or consists of an amino acid sequence having at least 75% identity, preferably at least 80% identity, more preferably at least 85% identity, most preferably at least 90% identity, and in particular at least 95% identity to the amino acid sequence of SEQ ID NO: 1, 2, 3, 4, 5, 6, 7, 8, 9, 10, 11, 12, 13, 14, 15, 16, 17, 18, 21, 22, 23, 24, 25, 26, 27, or 28; preferably SEQ ID NO: 1, 2, 8, 9, 11, 13, 17, 22, or 23. In preferred embodiments, the peroxygenase comprises or consists of an amino acid sequence having at least 70% identity, preferably at least 75% identity, more preferably at least 80% identity, more preferably at least 85% identity, most preferably at least 90% identity, and in particular at least 95% identity to the amino acid sequence of SEQ ID NO: 1. In more preferred embodiments, the peroxygenase comprises or consists of the amino acid sequence of SEQ ID NO: 1, 2, 3, 4, 5, 6, 7, 8, 9, 10, 11, 12, 13, 14, 15, 16, 17, 18, 21, 22, 23, 24, 25, 26, 27, or 28; preferably SEQ ID NO: 1, 2, 8, 9, 11, 13, 17, 22, or 23; or a fragment thereof having peroxygenase activity.

In other embodiments, the fatty acid has an even number of carbons from 4 to 28.

In other embodiments, a hydroxyl group is introduced in the fatty acid at position 2 or 3 of the terminal end.

In an embodiment of the second aspect, the lipoxygenase comprises or consists of an amino acid sequence having at least 70% identity, preferably at least 75% identity, more preferably at least 80% identity, more preferably at least 85% identity, most preferably at least 90% identity, and in particular at least 95% identity to the amino acid sequence of SEQ ID NO: 19. In a preferred embodiment, the lipoxygenase comprises or consists of the amino acid sequence of SEQ ID NO: 19.

In another aspect, the present invention provides a composition for hydroxylating an unsaturated fatty acid, or for bleaching the color of a carotenoid containing dye, stain, or composition, comprising a lipoxygenase and a peroxygenase. Preferably, the carotenoid is a carotene; more preferably the carotenoid is a lycopene or beta-carotene.

In an embodiment, the lipoxygenase has at least 70% identity to the amino acid sequence of SEQ ID NO: 19, and a peroxygenase having at least 70% identity to the amino acid sequence of SEQ ID NO: 1, 2, 3, 4, 5, 6, 7, 8, 9, 10, 11, 12, 13, 14, 15, 16, 17, 18, 21, 22, 23, 24, 25, 26, 27, or 28; preferably SEQ ID NO: 1, 2, 8, 9, 11, 13, 17, 22, or 23.

In an embodiment, the amino acid sequence of the peroxygenase comprises the motif: E-H-D-[G,A]-S-[L,I]-S-R (SEQ ID NO: 20).

In another embodiment, the composition includes a surfactant and/or is a detergent composition.

The present invention also provides for use of a peroxidase, as described above, for reducing the amount of fatty acid hydroperoxides in a fatty acid containing product; or for bleaching a carotenoid containing dye, stain, or composition. Preferably, the carotenoid is a carotene; more preferably the carotenoid is a lycopene or beta-carotene.

Similarly, the invention also provides for use of the composition of the invention for hydroxylating an unsaturated fatty acid.

The methods of the invention may be carried out with an immobilized peroxygenase.

The methods of the invention may be carried out in an aqueous solvent (reaction medium), various alcohols, ethers, other polar or non-polar solvents, or mixtures thereof. By studying the characteristics of the aliphatic hydrocarbon used in the methods of the invention, suitable examples of solvents are easily recognized by one skilled in the art. By raising or lowering the pressure at which the oxidation is carried out, the solvent (reaction medium) and the aliphatic hydrocarbon can be maintained in a liquid phase at the reaction temperature. The methods according to the invention may be carried out at a temperature between 0 and 90 degrees Celsius, preferably between 5 and 80 degrees Celsius, more preferably between 10 and 70 degrees Celsius, even more preferably between 15 and 60 degrees Celsius, most preferably between 20 and 50 degrees Celsius, and in particular between 20 and 40 degrees Celsius.

The methods of the invention may employ a treatment time of from 10 seconds to (at least) 24 hours, preferably from 1 minute to (at least) 12 hours, more preferably from 5 minutes to (at least) 6 hours, most preferably from 5 minutes to (at least) 3 hours, and in particular from 5 minutes to (at least) 1 hour.

The present invention is further described by the following examples that should not be construed as limiting the scope of the invention.

### EXAMPLES

### Example 1

### Reduction of Fatty Acid Hydroperoxides Using Peroxygenase

Experiments were performed using soybean lipoxygenase to generate fatty acid hydroperoxides from linoleic acid. The formation of the hydroperoxide species was demonstrated by absorbance increase at 234 nm. A subsequently added peroxygenase was able to use the oxidized product as oxidant (instead of hydrogen peroxide). The target molecule of the peroxygenase oxidation may either be the fatty acid derivative itself or ABTS.

### Materials

### Enzymes

0.24 mg/ml Soybean lipoxygenase (see SEQ ID NO: 19)
2.90 mg/ml Peroxygenasel from Agrocybe aegerita (see SEQ ID NO: 1)
0.30 mg/ml Peroxygenase2 from Coprinopsis cinerea (see SEQ ID NO: 2)
0.08 mg/ml Peroxygenase3 from Humicola insolens (see SEQ ID NO: 11)

### Substrates

25 mM Linoleic acid stock solution:
Mix 140 mg linoleic acid and 280 mg Tween20 in 5 ml milliQ water to get a milky white emulsion. Clarify the solution by addition of 0.6 ml 1N NaOH. Add milliQ water to a final volume of 20 ml and store in 1 ml aliquots in −20° C. freezer.
10 mM ABTS:
Dissolve 54.7 mg 2,2′-Azino-bis(3-ethylbenzothiazoline-6-sulfonic acid) diammonium salt (ABTS) in 10 ml milliQ water.

### Buffer

Mix 100 ml 1M KH₂PO₄/K₂HPO₄ (pH 6.5), 800 ml milliQ water and 250 mg Tween 20. Adjust pH to 6.5 and add milliQ water to 1 L.

### Method

### Assay

A mixture of buffer and linoleic acid was prepared by adding 40 μl linoleic acid stock solution to 1.96 ml buffer. All subsequent steps were carried out at room temperature using UV-transparent microplates and a microplate reader.

### Step 1:

180 μl substrate-buffer mixture was mixed with 10 μl lipoxygenase and the progress of the reaction was monitored by measuring absorbance at 234 nm. The oxidation of linoleic acid was considered completed when the change in absorbance at 234 nm ceased. Controls without addition of lipoxygenase were included.

### Step 2:

10 μl peroxygenase sample was added to each well after the linoleic acid was completely oxidized in step 1, and the progress of the reaction was monitored by measuring absorbance at 234 nm. The reaction was considered completed when the change in absorbance at 234 nm ceased. Controls without added peroxygenase were included.

### Step 3:

20 μl ABTS was added to each well and the progress of the reaction was monitored by measuring absorbance at 405 nm. The reaction was considered completed when the change in absorbance at 405 nm ceased.

### Results

The observed end-point absorbances at the given wavelengths of each reaction step are shown in Table 1.

TABLE 1
Absorbance measurements from assay steps 1-3.

| | Step 1 | Step 2 | Step 3 |
|---|---|---|---|
| | (Absor- | (Absor- | (Absor- |
| | bance at | bance at | bance at |
| Enzymes | 234 nm) | 234 nm) | 405 nm) |
| Peroxygenase1 + lipoxygenase | 3.20 | 3.35 | 0.676 |
| Peroxygenase1/no lipoxygenase | 0.34 | 0.48 | 0.044 |
| Peroxygenase2 + lipoxygenase | 3.16 | 3.17 | 0.193 |
| Peroxygenase2/no lipoxygenase | 0.36 | 0.41 | 0.047 |
| Peroxygenase3 + lipoxygenase | 3.18 | 0.58 | 0.083 |
| Peroxygenase3/no lipoxygenase | 0.35 | 0.35 | 0.051 |
| Lipoxygenase/no peroxygenase | 3.18 | 3.17 | 0.081 |

### Example 2

### Bleaching of a Carotene Using Peroxygenase and Lipoxygenase

Experiments were performed using soybean lipoxygenase to generate fatty acid hydroperoxides from linoleic acid. When the formation of the hydroperoxide species was completed (demonstrated by a constant level in absorbance at 234 nm), then a solution containing beta-carotene was added. Subsequently peroxygenases were added and decolorization or bleaching was followed by measuring absorbance at 450 nm.

### Materials

### Enzymes

Soy bean lipoxygenase (Sigma L-7395) (see SEQ ID NO: 19)
Peroxygenase4 from Poronia punctata (see SEQ ID NO: 8)
Peroxygenase5 from Chaetomium virescens (see SEQ ID NO: 9)
Peroxygenase6 from Chaetomium globosum (see SEQ ID NO: 13)
Peroxygenase7 from Sclerotinia sclerotiorum (see SEQ ID NO: 17)
Peroxygenase8 from Daldinia caldariorum (see SEQ ID NO: 22)
Peroxygenase9 from Myceliophthora fergusii (see SEQ ID NO: 23)

### Substrates and Stock Solutions

Tween 20 stock:
875 mg Tween 20 is dissolved in 50 ml of deionized water.
10 mM Linoleic acid stock solution:
210 mg linoleic acid (Sigma L1268) was dispersed in 3 ml 0.5N NaOH and mixed with 12 ml Tween 20 stock solution for about 5 minutes until the solution was clear. 60 ml cold deionized water was added to a final volume of 75 ml. The linoleic acid stock solution was stored in freezer.

### Beta-Carotene Stock Solution:

11 mg of beta-carotene (Sigma C-9750) was dissolved 50 ml of acetone. The solution was prepared fresh every second day and was stored refrigerated and in the dark.

### Buffer Solutions:

100 mM phosphate buffer pH 6.5

### Sigma Soy Bean Lipoxygenase Stock Solution:

10 mg powder (L-7395) was dissolved in 10 ml 50 mM phosphate buffer pH 6.5 (corresponding to 0.25 mg enzyme protein/ml). This solution was further diluted 3.5 times in deionized water (corresponding to a concentration of 0.07 mg enzyme protein/ml)

### Peroxygenase Stock Solution:

A stock solution containing 0.04 mg enzyme protein/ml was made for all peroxygenases in deionized water.

### Assay

150 μl of 100 mM phosphate buffer pH 6.5 was added in microtiter wells. 20 μl deionized water was added. 10 mM Linoleic acid stock solution was diluted 4.4 times in de-ionized water and 10 μl of this was added to the reaction well. 30 μl of Sigma soy bean lipoxygenase stock solution was added. The reaction mixture was mixed and incubated for 20 minutes at 30° C. 30 μl of beta-carotene stock solution was added. 30 μl of peroxygenase stock solution was added to start the reaction. Total volume of the reaction mixture was 300 μl. A SpectraMax Plus 384 plate reader was applied (kinetics at 30° C. at 450 nm) using a 96 well microtitre plate from Nunc (no. 260836). Double determinations were made for each peroxygenase sample. Controls following de-colorization without peroxygenase were made as an average of 8 samples. Decolorization was followed over 3 minutes. % decolorization was calculated as: (OD450_{control}-OD450ₛₐₘₚₗₑ₎/OD450_{control}*100. Absorbance of the control varied depending on how fresh the beta-carotene stock solution was prepared.

TABLE 2
End-concentrations in the reaction mixture.

| Compound | Concentration | |
|---|---|---|
| Buffer | 50 | mM |
| Linoleic acid | 0.075 | mM |
| Lipoxygenase | mg enzyme protein/ml 0.007 | |
| Beta-carotene | 0.04 | mM |

| | |
|---|---|
| Peroxygenase | mg enzyme protein/ml 0.004 |

### Results

TABLE 3
Absorbance measurement at 450 nm after
0, 1, 2 and 3 minute incubation.

| | Peroxygenase | | |
|---|---|---|---|
| | Absorbance of | Absorbance of | % |
| incubation time | control | Sample | Decolorization |

Peroxygenase4

| | | | |
|---|---|---|---|
| 0 min | 0.42 | 0.36 | 15 |
| 1 min | 0.42 | 0.29 | 32 |
| 2 min | 0.43 | 0.28 | 35 |
| 3 min | 0.42 | 0.27 | 37 |

Peroxygenase5

| | | | |
|---|---|---|---|
| 0 min | 0.42 | 0.34 | 19 |
| 1 min | 0.42 | 0.18 | 57 |
| 2 min | 0.43 | 0.13 | 70 |
| 3 min | 0.42 | 0.11 | 75 |

Peroxygenase6

| | | | |
|---|---|---|---|
| 0 min | 0.42 | 0.37 | 13 |
| 1 min | 0.42 | 0.31 | 28 |
| 2 min | 0.43 | 0.29 | 32 |
| 3 min | 0.42 | 0.28 | 35 |

Peroxygenase7

| | | | |
|---|---|---|---|
| 0 min | 0.42 | 0.35 | 17 |
| 1 min | 0.42 | 0.32 | 24 |
| 2 min | 0.43 | 0.31 | 28 |
| 3 min | 0.42 | 0.30 | 30 |

Peroxygenase8

| | | | |
|---|---|---|---|
| 0 min | 0.39 | 0.27 | 30 |
| 1 min | 0.36 | 0.17 | 53 |
| 2 min | 0.33 | 0.13 | 61 |
| 3 min | 0.30 | 0.12 | 61 |

Peroxygenase9

| | | | |
|---|---|---|---|
| 0 min | 0.42 | 0.36 | 14 |
| 1 min | 0.42 | 0.30 | 29 |
| 2 min | 0.43 | 0.29 | 33 |
| 3 min | 0.42 | 0.28 | 34 |

All of the tested peroxygenases were capable of decolorizing beta-carotene under the given conditions. High degree of beta-carotene bleaching was observed using Peroxygenase5, and Peroxygenase8, with more than 60% decolorization.

1. A method for reducing the amount of fatty acid hydroperoxides in a fatty acid containing product, comprising contacting the fatty acid hydroperoxide with a peroxygenase.

2. (canceled)

3. A method for bleaching a carotenoid containing dye, stain, or composition, comprising contacting the carotenoid with a lipoxygenase, oxygen, and a peroxygenase.

4. The method of claim 1, wherein the peroxygenase comprises an amino acid sequence which has at least 70% identity to SEQ ID NO: 1, 2, 3, 4, 5, 6, 7, 8, 9, 10, 11, 12, 13, 14, 15, 16, 17, 18, 21, 22, 23, 24, 25, 26, 27, or 28.

5. The method of claim 1, wherein the amino acid sequence of the peroxygenase comprises the motif: E-H-D-[G,A]-S-[L,I]-S-R (SEQ ID NO: 20).

6. The method of claim 1, wherein the peroxygenase comprises or consists of an amino acid sequence having at least 80% identity to the amino acid sequence of SEQ ID NO: 1, 2, 3, 4, 5, 6, 7, 8, 9, 10, 11, 12, 13, 14, 15, 16, 17, 18, 21, 22, 23, 24, 25, 26, 27, or 28.

7. The method of claim 1, wherein the peroxygenase comprises or consists of an amino acid sequence having at least 80% identity to the amino acid sequence of SEQ ID NO: 1.

8. The method of claim 1, wherein the peroxygenase comprises or consists of the amino acid sequence of SEQ ID NO: 1, 2, 3, 4, 5, 6, 7, 8, 9, 10, 11, 12, 13, 14, 15, 16, 17, 18, 21, 22, 23, 24, 25, 26, 27, or 28; or a fragment thereof having peroxygenase activity.

9. The method of claim 1, wherein the fatty acid has an even number of carbons from 4 to 28.

10. The method of claim 1, wherein a hydroxyl group is introduced in the fatty acid at position 2 or 3 of the terminal end.

11. (canceled)

12. (canceled)

13. The method of claim 3, wherein the carotenoid is a carotene, preferably beta-carotene or lycopene.

14. The method of claim 3, wherein the carotenoid containing dye, stain, or composition is a tomato containing dye, stain, or composition; or a carrot containing dye, stain, or composition.

15. A composition for hydroxylating an unsaturated fatty acid or for bleaching a carotenoid containing dye, stain, or composition, comprising a lipoxygenase and a peroxygenase.

16. The composition of claim 15, wherein the lipoxygenase has at least 70% identity to the amino acid sequence of SEQ ID NO: 19, and the peroxygenase has at least 70% identity to the amino acid sequence of SEQ ID NO: 1, 2, 3, 4, 5, 6, 7, 8, 9, 10, 11, 12, 13, 14, 15, 16, 17, 18, 21, 22, 23, 24, 25, 26, 27, or 28.

17. The composition of claim 15, wherein the amino acid sequence of the peroxygenase comprises the motif: E-H-D-[G,A]-S-[L,I]-S-R (SEQ ID NO: 20).

18. The composition of claim 15, which includes a surfactant and/or is a detergent composition.
